(19) United States
(12) Patent Application Publication
(10) Pub. No.: US 2009/0043226 A1
(43) Pub. Date: Feb. 12, 2009

(54) Methods for Collection of Oral Fluid

(75) Inventor: Eugene E. Elwell, Mt. Laurel, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: OFT LABS, LLC, Warrington, PA (US)

(21) Appl. No.: 12/180,869

(22) Filed: Jul. 28, 2008

Related U.S. Application Data
(60) Provisional application No. 60/954,209, filed on Aug. 6, 2007.
(60) Provisional application No. 60/954,961, filed on Aug. 9, 2007.

(51) Int. Cl. A61B 5/00

(52) U.S. Cl. 600/582

(57) ABSTRACT

Methods for collection of oral fluid are provided. The methods generally comprise swabbing the mandibular gumline of a subject. The methods are useful for, among other things, obtaining oral fluid for use in diagnostic assays.

### CROSS-REFERENCE TO RELATED APPLICATIONS

This application claims priority of U.S. Provisional Application Serial No. 60/954,209, filed Aug. 6, 2007, and U.S. Provisional Application Serial No. 60/954,961, filed Aug. 9, 2007, which are incorporated herein by reference in their entirety.

### FIELD

This invention relates generally to the field of biomedical diagnostics. More specifically, the invention relates to methods for collecting oral fluid that utilize swabs and minimize saliva collection.

### BACKGROUND

Various publications, including patents, published applications, technical articles and scholarly articles are cited throughout the specification. Each of these cited publications is incorporated by reference herein, in its entirety.

Mounting evidence indicates that inorganic and chemicals, biomolecules, and the like that are present in the blood serum can pass through oral mucosa into the mouth. As such, oral fluid is an attractive target for the detection of such molecules for purposes of diagnostics. Oral fluid as a diagnostic tool is based, in part, on the premise that the concentration of the molecule of interest in the oral fluid can be accurately correlated with the concentration of the molecule in the blood serum.

Invasive screening of bodily fluids, for example, blood collection through arterial or venous puncture, is generally used as the medical standard when screening for heavy metal exposure, illicit drug use, cancer, infection, and the like. Collection of blood presents the obvious problems of patient fear or discomfort, and the safety of personnel that collect, handle, or analyze the blood. Accordingly, where possible, non-invasive screens that are safe while maintaining the accuracy and reliability of blood-based screens are desired.

Common non-invasive screens for various diagnostics include the collection and analysis of other bodily fluids such as urine, saliva, sputum, mucous, tears, sweat, and the like, as well as solid collection and screening such as with feces, hair, skin, nails, and the like. The analysis of such bodily fluids and solids, however, has met with limited success in many instances. For example, screening for lead exposure in urine, feces, hair, nails, and saliva have been deemed to be unreliable (Barbosa F et al. (2005) Environ. Health Perspect. 113:1669-74; Barbosa F et al.(2006) Arch. Toxicol. 80:633-7; Thaweboon S et al. (2005) Southeast Asian J. Trop. Med. Public Health. 36:1576-9; and, Koh D et al. (2003) Occup. Environ Med. 60:696-8).

Often, with respect to a particular analyte, the lack of proper standards, lack of reliable reference values for human populations, and low levels of analyte present in urine, saliva, feces, hair, and the like are significant barriers to further consideration of non-invasive, blood-less screening. With the advent and development of more sensitive analytical equipment and techniques, however, the analysis of bodily fluids collected non-invasively is likely to gain increasing acceptance and prominence as a reliable diagnostic. In particular, oral fluid can conceivably replace blood as the gold standard for many diagnostic tests. Thus, there is a need in the art for means to accurately, consistently, and reliably collect oral fluid in preparation for subsequent analyses.

### SUMMARY

The invention features methods for collecting oral fluid from a subject. In some aspects, the methods comprise rubbing a swab along the vestibular aspect of the gumline of the subject. The gumline can be from the upper or lower dental arcade. The methods can further comprise rotating the swab concurrently with the rubbing, and the swab may be rotated in one or more particular areas along the dental arcade. If the swab is rubbed back and forth along the gumline, it is possible to stop the rubbing at one or more locations along the gumline and rotate the swab in such locations. Similarly, rotation at one or more locations along the gumline can proceed prior to beginning the rubbing. In all cases, rubbing can continue after rotation.

The swab can be rubbed for any period of time suitable under the circumstances to achieve a sufficient quantity of oral fluid. In some aspects, the rubbing proceeds for at least about 15 seconds, and in some aspects, the rubbing proceeds for at least about 20 seconds.

The swab itself can be comprised of any material suitable in the art for the collection of oral fluid. For example, in some aspects the swab comprises a polymer of ethylene glycol and terephthalic acid. In some aspects, the swab comprises alginate, cotton, sponge, filter paper, calcium fibers, cellulose, polyurethane, polyester, or rayon.

In highly preferred embodiments, the methods further comprise measuring the concentration of at least one analyte of interest in the oral fluid. Such measuring can occur using any means suitable in the art for analyzing the analyte of interest.

### BRIEF DESCRIPTION OF THE DRAWINGS

FIG. 1 shows the correlation of the concentration of lead in oral fluid with the concentration of lead in blood serum. Twenty three pairs of samples were analyzed by ICP-MS for oral fluid lead and blood lead for each patient.

### DETAILED DESCRIPTION

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “swab” refers to any device suitable for collecting oral fluid formed from an absorbent material. Preferably, the absorbent material is attached to a holder, but the term swab, as used herein, includes free standing devices made from absorbent material that may be placed completely adjacent to the gumline to absorb the oral fluid located therein.

“Oral fluid” as used herein refers to the liquid normally present in the region of the oral cavity anterior to the teeth and gumline. While oral fluid usually contains some saliva, only relatively small amounts of the secretions of the salivary glands are present, mixed with gingival crevicular fluid and other secretions from the musosal surfaces of the cheek and gums. Thus, oral fluid is more akin to an ultrafiltrate of the plasma than common saliva. Depending on the method employed to gather the oral fluid and the oral health of the subject from whom the sample is gathered, a sample may contain cells from the collection site and even small amounts of blood (including both blood cells and plasma).

As used herein, “measure” or “determine” refers to any qualitative or quantitative determinations.

Oral fluid is distinct from saliva. It has been discovered in accordance with the present invention that oral fluid is in fact superior to saliva for purposes of detecting various analytes and correlating the concentration of analyte in the oral fluid with the concentration of the analyte in the blood serum. It has further been discovered that optimized oral fluid collection, with minimal saliva contamination, can be accomplished by swabbing the vestibular aspect of the mandibular gumline. Accordingly, the invention features methods for collection of oral fluid from a subject.

In the methods of the invention, the subject can be any animal, and is preferably a mammal such as a human, mouse, rat, cat, dog, horse, cow, donkey, sheep, or pig. Humans are most preferred.

In some preferred aspects, the oral fluid is obtained by means of a swab. The swab can be comprised of any material suitable in the art such as alginate, cotton, sponge, capillary matrix, filter paper, calcium fibers, cellulose-based materials, or synthetic polymers such as a polyurethane, polyester, rayon, or a polymer of ethylene glycol and terephthalic acid. Polymers of ethylene glycol and terephthalic acid (i.e., polyethylene terephthalate) sold under the trade name DACRON® (DuPont) are particularly suited to the present invention. The absorbent material of the swab can be impregnated with salts or a hypertonic solution to facilitate absorption. The absorbent material can also be impregnated with a flavorant to make the sampling more pleasant to the subject. The swab can be on the end of a holder or an applicator made from plastic, wood, aluminum, and the like, with synthetic materials such as plastic being preferred.

The time required to collect oral fluid from the subject can vary. In some aspects, the oral fluid collection device is to remain in the subject's mouth for at least about 5 seconds, preferably at least about 10 seconds, more preferably at least about 15 seconds, and more preferably at least about 20 seconds, and more preferably at least about 20 seconds. In some aspects, the oral fluid collection device can remain in the subject's mouth for at least about 1 minute, or more.

The individual collecting the oral fluid can observe the oral fluid collection device after removing it from the subject's mouth to determine if a sufficient quantity of oral fluid has been collected. If an insufficient quantity has been collected, the oral fluid collection device can be placed back into the subject's mouth and the collection process repeated as described and exemplified herein. Such secondary collection can proceed for at least about 5 seconds through several minutes. It is contemplated that the experience and judgment of the individual collecting the oral fluid can be relied upon to determine the number of collection attempts, and the time required to collect sufficient quantities of oral fluid. What is a sufficient quantity may depend on several factors that would be expected to be known and understood by those of skill in the art. For example, such factors may include the analyte that will be the subject of the subsequent analysis, the subject's age, the oral health of the subject, the overall health of the subject, and the like.

The oral fluid can be obtained in a clinical or laboratory setting, and can be obtained from the upper jaw, lower jaw, or both, and can be obtained from the front or back of the mouth, or both. Preferably, the fluid is collected from the vestibular surface (either buccal or labial portions, or both) of the gumline (i.e., the line of interface between the gums and the teeth) of either the upper or lower dental arcade. Collection from the lower dental arcade (i.e. along the mandibular gumline) is preferred. The oral fluid can also be obtained from the space between the teeth and the gums.

Oral fluid can preferably be collected by moving the collection device back and forth, including with a swirling motion, along the gumline between the front and back of the mouth, or between the left and right sides of the mouth. Since it is preferable that the percentage of gingival crevicular fluid in the oral fluid be high, the amount of saliva in the oral fluid should be kept to a minimum. Thus, collection procedures that stimulate saliva production should be avoided. Such techniques are well known to those of skill in the art.

The oral fluid sample can be analyzed shortly after collection, or can be stored for later analysis. The oral fluid sample may also be frozen for long-term storage. Upon long-term storage, a suitable preservative or stabilizer can be added to the oral fluid sample, so long as the preservative or stabilizer does not introduce heavy metal contamination in a way that would skew the analysis. The sample can be analyzed with the oral fluid in its liquid form, or the sample can be dried prior to analysis. Dried samples can be stored indefinitely.

The oral fluid can be subsequently analyzed using any technique suitable in the art for detection and/or quantification of the analyte of interest. Non-limiting examples of applications in which oral fluid can be analyzed include screening for evidence of alcohol, drug, or hormone use, abuse, or overdose, for heavy metal exposure, for the presence of biological metabolites, for cancer, for microbial or vial infection, and the like.

Drugs that can be screened by way of oral fluid collection and analysis include, but are not limited to, tetrahydrocannabinol (THC) and its metabolites, cocaine, opiates, phencyclidine, amphetamines, methamphetamines, methylenedioxymethamphetamine, methaqualone, benzodiazepines, steroids, barbiturates, hallucinogens such as LSD and psilocybin, heroin, methadone, oxycodone, methylphenidate, rohypnol, leva alpha acetyl methadol, fentanyl, nicotine, contine, and the like. Heavy metals that can be screened include without limitation antimony, arsenic, beryllium, bismuth, cadmium, cerium, chromium, cobalt, copper, gallium, gold, iron, lead, manganese, mercury, nickel, platinum, silver, tellurium, thallium, tin, vanadium, and zinc. Non-limiting examples of hormones that can be screened in oral fluid include human growth hormone, androstenediol, androstenedione, boldenone, clostebol, dehydrochloromethyl-testosterone, dehydroepiandrosterone, dihydrotestosterone, dromostanolone, epitrenbolone trenbolone, fluoxymesterone, gestrinone, mesterolone, methandienone, methenolone clenbuterol, methyltestosterone, mandrolone, norandrostenediol, norandrostenedione, norethandrolone, oxandrolone, oxymetholone, stanozolol, tetrahydrogestinone, estrogen, progesterone, luteinizing hormone, thyroid hormones (T4-free, T4 total, T3), thyroid stimulating hormone, corticotrophin, human chorionic gonadotropin, somatotrophin, insulin like growth hormone, and the like. Alcohols such as methanol, ethanol, butanol, and isopropanol can also be detected from oral fluid. In addition, biologicals such as phytoestrogen, terpenoids, ascorbates, and alkaloids can also be screened from oral fluid samples.

The following examples are provided to describe the invention in greater detail. They are intended to illustrate, not to limit, the invention.

### EXAMPLE 1

### Determination of the Weight Variability in Polyester Swabs

For many applications involving the analysis of oral fluid collected by swabbing, the swabs containing oral fluid are dried prior to analysis. Drying of the swab prior to analysis eliminates extent of evaporation as a variable. According to this technique, the swab is separated from the holder at the base of the swab material, dried, then weighed using a high accuracy analytical balance. The mass of a corresponding blank swab is then subtracted from mass obtained, to determine the mass of sample in the swab.

It is thus important to select swabs that are of a consistent mass, at least within a particular manufacturing lot. One swab that meets the requirements of this analytical method is marketed by Copan Innovation. The dry weight of these sterile ethylene glycol/terephthalic acid swabs was determined using an analytical balance that is accurate to 10-4 grams. Ninety four swabs were weighed, and determined to have an average mass of 0.0934 g, with a standard deviation calculated to be 0.0033. Thus, very little variation in the mass of this brand of swab was observed.

Prior to analysis, the swabs can be digested to reduce interference by contaminants, and in some applications, to convert an analyte associated with particulates to a form that can be analyzed using a particular analytical technique such as inductively-coupled plasma spectrometry (ICP-MS). Microwave digestion can be used as an alternative. The microwave method is a closed-vessel procedure and thus provides improved precision.

Oral fluid samples are prepared on a weight basis. Thus, acid blanks are prepared in parallel for each type of digestion performed. Although it is always best to eliminate all relevant sources of contamination, a reagent blank prepared with the same acids and subjected to the same digestion procedure as the sample can correct for impurities present in acids and reagent water.

### EXAMPLE 2

### Analysis of Oral Fluid Samples for the Presence of Lead

Dried Dacron® swabs from Copan Innovation, containing oral fluid samples, were excised from the handle at the proximal extent of the fibers and weighed. The mass of blank swabs from the same lot were subtracted, to determine the dry weight of the sample. The sample swabs were then each dissolved in 1 ml of nitric acid, and diluted to a final volume of 15 ml with 18 megaohm deionized water, to which indium (¹¹⁵In) was added to a concentration of 20 parts per billion. An unused swab was prepared in parallel to serve as a negative control. Samples were then assayed by ICP-MS on a Perkin Elmer Elan Dynamic Reaction Cell™ II ICP-MS or a PerkinElmer ELAN 9000 ICP-MS.

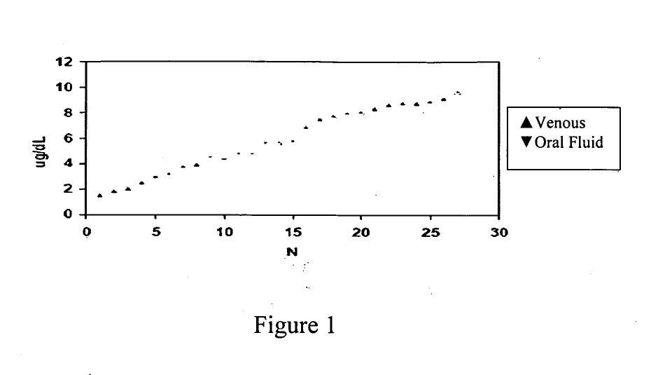

Patient samples of oral fluid and blood were analyzed in parallel using inductively couple plasma mass spectrometry to determine the concentration of lead in each respective biological fluid. Statistical analyses were carried out to determine whether the level of lead in each fluid could be correlated. The results are provided in FIG. 1. Ninety five children were tested for lead (mean 3.3 mg/dL+1.3 mg/dL, range 3 mg/dL−45 mg/dL). The correlation between oral fluid and blood lead concentration was significant (0.879, p<0.05). Of twenty one test between 3 mg/dL and 10 mg/dL, the correlation was 0.938 (p<0.001, two tailed) between blood and oral fluid.

What is claimed:

1. A method for collecting oral fluid from a subject, comprising rubbing a swab along the vestibular aspect of the gumline of the subject.

2. The method of claim 1 wherein the swab comprises a polymer of ethylene glycol and terephthalic acid.

3. The method of claim 1, wherein the swab comprises alginate, cotton, sponge, filter paper, calcium fibers, cellulose, polyurethane, polyester, or rayon.

4. The method of claim 1, further comprising rotating the swab concurrently with the rubbing.

5. The method of claim 1, comprising rubbing the swab for at least about fifteen seconds.

6. The method of claim 1, comprising rubbing the swab for at least about twenty seconds.

7. The method of claim 1, wherein the swab is impregnated with a salt or hypertonic solution.

8. The method of claim 1, wherein the swab is rubbed along the gumline of the upper dental arcade.

9. The method of claim 1, wherein the swab is rubbed along the gumline of the lower dental arcade.

10. The method of claim 1, wherein the subject is a human.

11. The method of claim 1, further comprising measuring the concentration of at least one analyte of interest in the oral fluid.
